(19) United States
(12) Patent Application Publication
(10) Pub. No.: US 2021/0113172 A1
(43) Pub. Date: Apr. 22, 2021

(54) Lesion Detection Method, Apparatus and Device, and Storage Medium

(71) Applicant: Beijing Sensetime Technology Development Co., Ltd., Beijing (CN)

(72) Inventors: Rui Huang, Beijing (CN); Yunhe Gao, Beijing (CN)

(73) Assignee: Beijing Sensetime Technology Development Co., Ltd., Beijing (CN)

(21) Appl. No.: 17/134,771

(22) Filed: Dec. 28, 2020

Related U.S. Application Data
(63) Continuation of application No. PCT/CN2019/114452, filed on Oct. 30, 2019.

(30) Foreign Application Priority Data
Dec. 7, 2018 (CN) 201811500631.4

(51) Int. Cl. A61B 6/00; G06T 7/73; G06T 3/40; G06T 7/00; A61B 6/03; G16H 30/40; G16H 50/20; G16H 15/00; G06N 3/04; G06N 3/08

(52) U.S. Cl. CPC A61B 6/5223; G06T 2207/10081; G06T 3/4046; G06T 7/0012; A61B 6/032; A61B 6/5217; A61B 6/463; G16H 30/40; G16H 50/20; G16H 15/00; G06N 3/0454; G06N 3/08; G06T 2207/30096; G06T 2207/20084; G06T 2207/20081; G06T 7/73

(57) ABSTRACT

The present disclosure discloses a lesion detection method and apparatus, device, and a storage medium. The method includes: obtaining a first image comprising a plurality of sample slices, the first image being a three-dimensional image; performing feature extraction on the first image, to generate a first feature graph comprising a feature and a position of a lesion; the first feature graph comprising a three-dimensional feature; performing dimension reduction processing on the feature comprised in the first feature graph, to generate a second feature graph; the second feature graph comprising a two-dimensional feature; and detecting the second feature graph, to obtain a position of each lesion in the second feature graph and a confidence score corresponding to the position. Using the present disclosure may accurately detect lesion conditions of multiple parts in a patient body, and implement cancer preliminary assessment of a full body range of the patient.

### CROSS REFERENCE TO RELATED APPLICATIONS

The present application is a continuation of and claims priority under 35 U.S.C. 120 to PCT Application. No. PCT/CN2019/114452, filed on Oct. 30, 2019, and entitled “METHOD, APPARATUS AND DEVICE FOR DETECTING LESION, AND STORAGE MEDIUM”, which claims priority to Chinese Patent Application No. 201811500631.4, filed to the Chinese Patent Office on Dec. 7, 2018 and entitled “LESION DETECTION METHOD, APPARATUS AND DEVICE”. All above-referenced priority documents are incorporated herein by reference in their entirety.

### TECHNICAL FIELD

The present disclosure relates to the field of computer technologies, and in particular, to lesion detection methods and apparatus, devices, and storage media.

### BACKGROUND

A Computer Aided Diagnosis (CAD) refers to automatically discovering a lesion in an image by means of iconography and medical image analysis technology and other possible physiological and biochemical means in combination of analysis and computing of a computer. Practice has proven that the CAD has played a very positive role in promoting aspects such as improving diagnosis accuracy, reducing missed diagnosis, and improving doctor working efficiency. Lesion refers to a site of the lesion due to an action of pathogenic factors on tissues or organs, and is a part on a body suffered from pathological changes. For example, a part of a lung of a body is destroyed by tubercle bacillus, and then this part is a tuberculosis (TB) lesion.

In recent years, as the rapid development of computer vision and depth learning technologies, the CT image based lesion detection method receives more and more attention.

### SUMMARY

The present disclosure provides a lesion detection method, apparatus, and device and a storage medium, which may accurately detect lesion conditions of multiple parts in a patient body, and implement cancer preliminary assessment of a full body range of the patient.

For a first aspect, the present disclosure provides a lesion detection method. The method includes: obtaining a first image comprising a plurality of sample slices, the first image being a three-dimensional image comprising an X axis dimension, a Y axis dimension, and a Z axis dimension; performing feature extraction on the first image, to generate a first feature graph comprising a feature and a position of a lesion; the first feature graph comprising a three-dimensional feature comprising an X axis dimension, a Y axis dimension, and a Z axis dimension; performing dimension reduction processing on the feature comprised in the first feature graph, to generate a second feature graph; the second feature graph comprising a two-dimensional feature of the X axis dimension and the Y axis dimension; and detecting the second feature graph, to obtain a position of each lesion in the second feature graph and a confidence score corresponding to the position.

By combining the first aspect, in some possible embodiments, the obtaining the first image comprising the plurality of sample slices comprises: resampling an obtained CT image of a patient at a first sampling interval, to generate the first image comprising the plurality of sample slices.

By combining the first aspect, in some possible embodiments, the performing feature extraction on the first image, to generate the first feature graph comprising the feature and the position of the lesion, includes: downsampling the first image by means of a first neural network, to generate a third feature graph; downsampling the third feature graph by means of a residual module of the second neural network, to generate a fourth feature graph; extracting features of lesions having different dimensions in the fourth feature graph by means of a DenseASPP module of the second neural network; after processing by the DenseASPP module, generating a fourth preset feature graph having a same resolution ratio as the fourth feature graph, and upsampling the feature graph processed by the DenseASPP module by means of a deconvolutional layer and the residual module of the second neural network, to generate a third preset feature graph having a same resolution ratio as the third feature graph; and generating a first feature graph having a same resolution ratio as the third preset feature graph from the third feature graph and the third preset feature graph, and fusing the four feature graph and the fourth preset feature graph, to generate a first feature graph having a same resolution ratio as the fourth preset feature graph; the third preset feature graph and the fourth preset feature graph respectively comprising the position of the lesion; and the position of the lesion being used for generating the position of the lesion in the first feature graph.

By combining the first aspect, in some possible embodiments, the performing feature extraction on the first image, to generate the first feature graph comprising the feature and the position of the lesion, includes: downsampling the first image by means of a residual module of the second neural network, to generate a fourth feature graph; extracting features of lesions having different dimensions in the fourth feature graph by means of a DenseASPP module of the second neural network; after processing by the DenseASPP module, upsampling the feature graph processed by the DenseASPP module by means of the deconvolutional layer and the residual module of the second neural network, to generate the first preset feature graph having a same resolution ratio as the first image; and

generating the first feature graph having a same resolution ratio as the first preset feature graph from the first image and the first preset feature graph; the first preset feature graph comprising the position of the lesion; and the position of the lesion being used for generating the position of the lesion in the first feature graph.

By combining the first aspect, in some possible embodiments, the performing feature extraction on the first image, to generate the first feature graph including the feature and the position of the lesion, includes: downsampling the first image by means of a first neural network, to generate a third feature graph; downsampling the third feature graph by means of a residual module of the second neural network, to generate a fourth feature graph; downsampling the fourth feature graph by means of the residual module of the second neural network, to generate a fifth feature graph having a resolution ratio less than the fourth feature graph; extracting features of lesions having different dimensions in the fifth feature graph by means of the DenseASPP module of the second neural network; after processing by the DenseASPP module, generating a fifth preset feature graph having a same resolution ratio as the fifth feature graph, and upsampling the feature graph processed by the DenseASPP module by means of the deconvolutional layer and the residual module of the second neural network, to generate a fourth preset feature graph having a same resolution ratio as the fourth feature graph; or upsampling the feature graph processed by the DenseASPP module by means of the deconvolutional layer and the residual module of the second neural network, to generate a third preset feature graph having a same resolution ratio as the third feature graph; and fusing the third feature graph and the third preset feature graph, to generate the first feature graph having a same resolution ratio as the third preset feature graph; fusing the fourth feature graph and the fourth preset feature graph, to generate the first feature graph having a same resolution ratio as the fourth preset feature graph; and fusing the fifth feature graph and the fifth preset feature graph, to generate the first feature graph having a same resolution ratio as the fifth preset feature graph; the third preset feature graph, the fourth preset feature graph, and the fifth preset feature graph respectively including the position of the lesion; and the position of the lesion being used for generating the position of the lesion in the first feature graph.

By combining the first aspect, in some possible embodiments, the first neural network includes: a convolutional layer and a residual module cascaded with the convolutional layer; and the second neural network includes: a 3D U-Net network; the 3D U-Net network includes: a convolutional layer, a deconvolutional layer, a residual module, and the DenseASPP module.

By combining the first aspect, in some possible embodiments, the second neural network is a plurality of stacked 3D U-Net networks.

By combining the first aspect, in some possible embodiments, the residual module includes: a convolutional layer, a batch normalization layer, a ReLU activation function, and a maximum pooling layer.

By combining the first aspect, in some possible embodiments, the performing dimension reduction processing on the feature comprised in the first feature graph, to generate the second feature graph, includes: respectively combining a channel dimension and Z axis dimension of each feature in all features of the first feature graph, so that the dimension of each feature in all features of the first feature graph consists of the X axis dimension and the Y axis dimension; where the first feature graph with the dimension of each feature in all the features consisting of the X axis dimension and the Y axis dimension is the second feature graph.

By combining the first aspect, in some possible embodiments, the detecting the second feature graph includes: detecting the second feature graph by means of a first detection sub-network, to obtain a coordinate of the position of each lesion in the second feature graph by means of detection; and detecting the second feature graph by means of a second detection sub-network, to obtain a confidence score corresponding to each lesion in the second feature graph by means of detection.

By combining the first aspect, in some possible embodiments, the first detection sub-network includes: a plurality of convolutional layers, each of the plurality of convolutional layers being connected to a ReLU activation function; and the second detection sub-network includes: a plurality of convolutional layers, each of the plurality of convolutional layers being connected to a ReLU activation function.

By combining the first aspect, in some possible embodiments, before the performing feature extraction on the first image, to generate the first feature graph including the feature and the position of the lesion, further including: inputting a pre-stored three-dimensional image including a plurality of lesion annotations into the first neural network; the lesion annotations being used for annotation of the lesions; and using a gradient descent method to respectively train each parameter of the first neural network, the second neural network, the first detection sub-network, and the second detection sub-network, where the position of each lesion in the plurality of lesions are output by the first detection sub-network.

By combining the first aspect, in some possible embodiments, before the performing feature extraction on the first image, to generate the first feature graph including the feature and the position of the lesion, further including: inputting a pre-stored three-dimensional image including a plurality of lesion annotations into the second neural network; the lesion annotations being used for annotation of the lesions; and using a gradient descent method to respectively train each parameter of the second neural network, the first detection sub-network, and the second detection sub-network, where the position of each lesion in the plurality of lesions are output by the first detection sub-network.

For a second aspect, the present disclosure provides a lesion detection apparatus. The apparatus includes: an obtaining unit, configured to obtain a first image including a plurality of sample slices, the first image being a three-dimensional image including an X axis dimension, a Y axis dimension, and a Z axis dimension; a first generation unit, configured to perform feature extraction on the first image, to generate a first feature graph including a feature and a position of a lesion; the first feature graph including a three-dimensional feature including an X axis dimension, a Y axis dimension, and a Z axis dimension; a second generation unit, configured to perform dimension reduction processing on the feature comprised in the first feature graph, to generate a second feature graph; the second feature graph including a two-dimensional feature of the X axis dimension and the Y axis dimension; and a detection unit, configured to detect the second feature graph, to obtain a position of each lesion in the second feature graph and a confidence score corresponding to the position.

By combining the second aspect, in some possible embodiments, the obtaining unit is specifically configured to: resample an obtained CT image of a patient at a first sampling interval, to generate the first image including the plurality of sample slices.

By combining the second aspect, in some possible embodiments, the first generation unit is specifically configured to: downsample the first image by means of a first neural network, to generate a third feature graph; downsample the third feature graph by means of a residual module of the second neural network, to generate a fourth feature graph; extract features of lesions having different dimensions in the fourth feature graph by means of a DenseASPP module of the second neural network; after processing by the DenseASPP module, generate a fourth preset feature graph having a same resolution ratio as the fourth feature graph, and upsample the feature graph processed by the DenseASPP module by means of a deconvolutional layer and the residual module of the second neural network, to generate a third preset feature graph having a same resolution ratio as the third feature graph; and generate a first feature graph having a same resolution ratio as the third preset feature graph from the third feature graph and the third preset feature graph, and fuse the four feature graph and the fourth preset feature graph, to generate a first feature graph having a same resolution ratio as the fourth preset feature graph; the third preset feature graph and the fourth preset feature graph respectively including the position of the lesion; and the position of the lesion being used for generating the position of the lesion in the first feature graph.

By combining the second aspect, in some possible embodiments, the first generation unit is specifically configured to: downsample the first image by means of a residual module of the second neural network, to generate a fourth feature graph; extract features of lesions having different dimensions in the fourth feature graph by means of a DenseASPP module of the second neural network; after processing by the DenseASPP module, upsampling the feature graph processed by the DenseASPP module by means of the deconvolutional layer and the residual module of the second neural network, to generate the first preset feature graph having a same resolution ratio as the first image; and generate the first feature graph having a same resolution ratio as the first preset feature graph from the first image and the first preset feature graph; the first preset feature graph including the position of the lesion; and the position of the lesion being used for generating the position of the lesion in the first feature graph.

By combining the second aspect, in some possible embodiments, the first generation unit is specifically configured to: downsample the first image by means of the first neural network, to generate the third feature graph having the resolution ratio less than the first image; downsample the third feature graph by means of a residual module of the second neural network, to generate a fourth feature graph; downsample the fourth feature graph by means of a residual module of the second neural network, to generate a fifth feature graph; extract features of lesions having different dimensions in the fifth feature graph by means of the DenseASPP module of the second neural network; after processing by the DenseASPP module, generate a fifth preset feature graph having a same resolution ratio as the fifth feature graph, and upsample the feature graph processed by the DenseASPP module by means of the deconvolutional layer and the residual module of the second neural network, to generate a fourth preset feature graph having a same resolution ratio as the fourth feature graph; or upsample the feature graph processed by the DenseASPP module by means of the deconvolutional layer and the residual module of the second neural network, to generate a third preset feature graph having a same resolution ratio as the third feature graph; and fuse the third feature graph and the third preset feature graph, to generate the first feature graph having a same resolution ratio as the third preset feature graph; fuse the fourth feature graph and the fourth preset feature graph, to generate the first feature graph having a same resolution ratio as the fourth preset feature graph; and fuse the fifth feature graph and the fifth preset feature graph, to generate the first feature graph having a same resolution ratio as the fifth preset feature graph; the third preset feature graph, the fourth preset feature graph, and the fifth preset feature graph respectively including the position of the lesion; and the position of the lesion being used for generating the position of the lesion in the first feature graph.

By combining the second aspect, in some possible embodiments, the first neural network includes: a convolutional layer and a residual module cascaded with the convolutional layer; and the second neural network includes: a 3D U-Net network; the 3D U-Net network includes: a convolutional layer, a deconvolutional layer, a residual module, and the DenseASPP module.

By combining the second aspect, in some possible embodiments, the second neural network is a plurality of stacked 3D U-Net networks.

By combining the second aspect, in some possible embodiments, the residual module includes: a convolutional layer, a batch normalization layer, a ReLU activation function, and a maximum pooling layer.

By combining the second aspect, in some possible embodiments, the third feature unit is specifically configured to respectively combine a channel dimension and Z axis dimension of each feature in all features of the first feature graph, so that the dimension of each feature in all features of the first feature graph consists of the X axis dimension and the Y axis dimension; where the first feature graph with the dimension of each feature in all the features consisting of the X axis dimension and the Y axis dimension is the second feature graph.

By combining the second aspect, in some possible embodiments, the detection unit is specifically configured to: detect the second feature graph by means of a first detection sub-network, to obtain a coordinate of the position of each lesion in the second feature graph by means of detection; and detect the second feature graph by means of a second detection sub-network, to obtain a confidence score corresponding to each lesion in the second feature graph by means of detection.

By combining the second aspect, in some possible embodiments, the first detection sub-network includes: a plurality of convolutional layers, each of the plurality of convolutional layers being connected to a ReLU activation function; and the second detection sub-network includes: a plurality of convolutional layers, each of the plurality of convolutional layers being connected to a ReLU activation function.

By combining the second aspect, in some possible embodiments, a training unit is further included, and is specifically configured to: before the first generation unit performs feature extraction on the first image to generate a first feature graph including a feature of a lesion, input a pre-stored three-dimensional image including a plurality of lesion annotations into the first neural network; the lesion annotations being used for annotation of the lesions; and use a gradient descent method to respectively train each parameter of the first neural network, the second neural network, the first detection sub-network, and the second detection sub-network, where the position of each lesion in the plurality of lesions are output by the first detection sub-network.

By combining the second aspect, in some possible embodiments, a training unit is further included, and is specifically configured to: before the first generation unit performs feature extraction on the first image to generate a first feature graph including a feature and a position of a lesion, input a three-dimensional image including a plurality of lesion annotations into the second neural network; the lesion annotations being used for annotation of the lesions; and use a gradient descent method to respectively train each parameter of the second neural network, the first detection sub-network, and the second detection sub-network, where the position of each lesion in the plurality of lesions are output by the first detection sub-network.

For a third aspect, the present disclosure provides a lesion detection device, including: a display, a memory, and a processor connected with one another, where the display is configured to display a position of a lesion and a confidence score corresponding to the position, the memory is configured to store application program codes, and the processor is configured to invoke the program codes, to execute the lesion detection method according to the aforementioned first aspect.

For a fourth aspect, the present disclosure provides a computer readable storage medium for storing one or more computer programs, the aforementioned one or more computer program include program instructions, and when the computer programs run on the computer, the aforementioned instructions are used for executing the lesion detection method according to the aforementioned first aspect.

For a fifth aspect, the present disclosure provides a computer program, including lesion detection instructions, where when the computer program runs on the computer, the aforementioned lesion detection instructions are used for implementing the lesion detection method according to the aforementioned first aspect.

The present disclosure provides a lesion detection method, apparatus, and device, and a storage medium. First, a first image comprising a plurality of sample slices is obtained, and the first image is a three-dimensional image comprising an X axis dimension, a Y axis dimension, and a Z axis dimension. Second, feature extraction is performed on the first image, to generate a first feature graph comprising a feature and a position of a lesion; the first feature graph includes a three-dimensional feature comprising an X axis dimension, a Y axis dimension, and a Z axis dimension. Then, dimension reduction processing is performed on the feature included in the first feature graph, to generate a second feature graph; and the second feature graph includes a two-dimensional feature of the X axis dimension and the Y axis dimension. Finally, the feature of the second feature graph is detected obtain a feature of each lesion in the second feature graph and a confidence score corresponding to the position. Using the present disclosure may accurately detect lesion conditions of multiple parts in a patient body, and implement cancer preliminary assessment of a full body range of the patient.

### BRIEF DESCRIPTION OF THE DRAWINGS

To describe the technical solutions in embodiments of the present disclosure more clearly, the accompanying drawings required for describing the embodiments are briefly introduced below. Apparently, the accompanying drawings in the following description show some embodiments of the present disclosure, and a person of ordinary skill in the art can still derive other accompanying drawings from these accompanying drawings without creative efforts.

FIG. 1 is a schematic diagram of a network framework of a lesion detection system provided by the present disclosure.

FIG. 2 is a schematic flowchart of a lesion detection method provided by the present disclosure.

FIG. 3 is a schematic box diagram of a lesion detection apparatus provided by the present disclosure.

FIG. 4 is a structural schematic diagram of a lesion detection apparatus provided by the present disclosure.

### DETAILED DESCRIPTION

The technical solutions of the present disclosure are described clearly and integrally by combining the drawing in the disclosure; obviously, the described embodiments are some of the embodiments of the present disclosure rather than all the embodiments. Based on the embodiments of the present disclosure, all other embodiments obtained by a person of ordinary skill in the art without involving an inventive effort shall fall within the scope of protection of the present disclosure.

It should be understood that the terms “include” and “comprise”, when used in the present description and the appended claims, specify the presence of stated features, entireties, steps, operations, elements, and/or assemblies, but do not preclude the presence or addition of one or more other features, entireties, steps, operations, elements, assemblies, and/or combinations thereof.

It should be further understood that here the terms used in the description of the present disclosure are merely intended for describing particular embodiments other than limiting the present disclosure. As used in the description and appended claims of the present disclosure, the singular forms “a”, “an”, and “the” are intended to include the plural forms, unless expressly stated otherwise.

It should be further understood that the term “and/or” used in the description and appended claims of the present disclosure indicate associating with any combination and all possible combinations of one or more listed items, and including all the combinations.

As used in the present description and the appended claims, the term “if” can be interpreted as “when” or “once” or “in response to determining” or “in response to detecting” according to the context. Similarly, the phase “if determining” or “if detecting [a described condition or event]” can be interpreted as “once determining” or “in response to determining” or “once detecting [a described condition or event]” or “in response to detecting [a described condition or event]” according to the context.

In specific implementations, the device described in the present disclosure includes, but not limited to, for example, a laptop or a tablet computer having a touch sensitive surface (for example, a touch screen display and/or a touch tablet), and other portable devices. It should be further understood that in some embodiments, the device is not a portable communication device, but a desk computer having a touch sensitive surface (for example, a touch screen display and/or a touch tablet).

In the following discussions, the device including a display and a touch sensitive surface is described. However, it should be understood that the device may include one or more other physical user interface devices, such as a physical keyboard, a mouse, and/or a joystick.

The device supports various application programs, for example, one or more of the following: a drawing application program, a demonstration application program, a character processing application program, a network creating application program, a disc recording application program, an electronic table application program, a game application program, a telephone application program, a video conference application program, an E-mail application program, an instant message application program, an exercise support application program, a photo management application program, a digital camera application program, a digital video camera application program, a web browsing application program, a digital music player application program, and/or a digital video player application program.

The various application programs that can be executed on the device may use at least a common physical user interface device of a touch sensitive surface. One or more functions of the touch sensitive surface and corresponding information displayed on the device may be adjusted and/or changed among the application programs and/or in the corresponding application program. In this way, the common physical framework (for example, the touch sensitive surface) of the device may support various application programs of the user interface which is visual and transparent to the user.

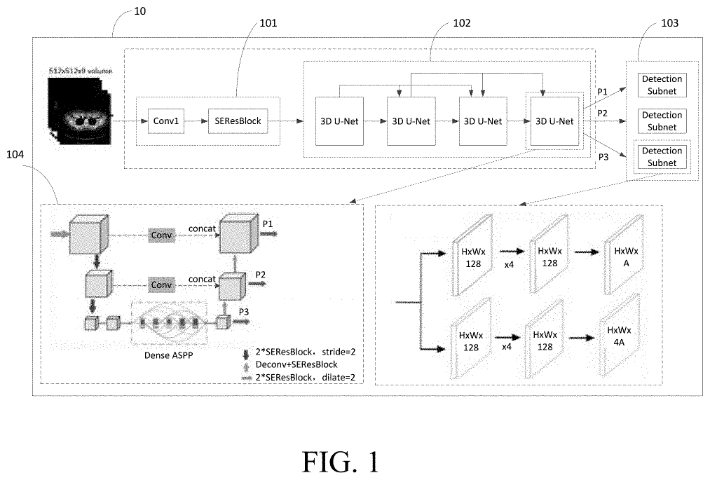

For better understanding the present disclosure, the network framework adapted to the present disclosure is described as follows. Referring to FIG. 1, FIG. 1 is a schematic diagram of a lesion detection system provided by the present disclosure. As shown in FIG. 1, a system 10 may include: a first neural network 101, a second neural network 102, and a detection sub-network 103.

In the embodiments of the present disclosure, the lesion refers to a site of the lesion due to an action of pathogenic factors on tissues or organs, and is a part on a body suffered from pathological changes. For example, a part of a lung of a body is destroyed by tubercle bacillus, and then this part is a tuberculosis (TB) lesion.

It should be explained that the first neural network 101 includes a convolutional layer (Conv1) and a residual module (SEResBlock) cascaded with the convolutional layer. The residual module may include a Batch Normalization (BN) layer, a ReLU activation function, and a maximum pooling layer (Max-pooling).

The first neural network 101 may be used for downsampling the first image input into the first neural network 101 at the X axis dimension and the Y axis dimension. It should be explained that the first image is a three-dimensional image including an X axis dimension, a Y axis dimension, and a Z axis dimension (that is, the first image is a three-dimensional image including an X axis dimension, a Y axis dimension, and a Z axis dimension constituted by two-dimensional images including an X axis dimension and a Y axis dimension), for example, the first image may be a three-dimensional image of 512*512*9. Specifically, the first neural network 101 processes the first image by means of convolution kernel generation in the convolutional layer, to generate a feature graph, and furthermore, the first neural network 101 pools the specific feature graph by means of a residual module, so as to generate a third feature graph having a smaller resolution ratio than the first image. For example, the three-dimensional image of 512*512*9 may be processed by means of the first neural network 101 into a three-dimensional image of 256*256*9, or the three-dimensional image of 512*512*9 may further be processed by means of the first neural network 101 into a three-dimensional image of 128*128*9. The downsampling process may extract the lesion feature included in the input first image and remove same unnecessary regions in the first image.

In should be explained that the purpose of the downsampling in the embodiments of the present disclosure is to generate a thumbnail of the first image so that the first image complies with the size of the display region. The purpose of the upsampling in the embodiments of the present disclosure is to insert new pixels by means of interpolated values among pixels in an original image to amplify the original image. It facilitates the detection of small lesions.

An example is exemplified below to make a simple explanation for the downsampling in the embodiments of the present disclosure. For example, for an image I with a size of M*N, S times of downsampling is performed on the image I, to obtain the image with the resolution ratio of the (M/S)*(N/S) size. That is, the image in an S*S window in the original image I is changed into a pixel, wherein the pixel value of the pixel is the maximum value of all pixels in the S*S window. A stride sliding along a horizontal direction or a vertical direction may be 2.

The second neural network 102 may include four stacked 3D U-net networks. The expanded view of the 3D U-net networks is 104 shown in FIG. 1. Multiple detections of the 3D U-net networks may improve the accuracy of the detection; the embodiments of the present disclosure only exemplify the number of the 3D U-net network, not limit same. The 3D U-net network includes a convolutional layer, a deconvolutional layer, a residual module, and a DenseASPP module.

The residual module of the second neural network 102 may be used for downsampling the third feature graph output by the first neural network 101 at the X axis dimension and the Y axis dimension, to generate a fourth feature graph.

In addition, the residual module of the second neural network 102 may also be used for downsampling the fourth feature graph at the X axis dimension and the Y axis dimension, to generate a fifth feature graph.

Next, features of lesions having different dimensions in the fifth feature graph are extracted by means of the DenseASPP module of the second neural network 102.

After processing by the DenseASPP module, a fifth preset feature graph having a same resolution ratio as the fifth feature graph is generated, and the feature graph processed by the DenseASPP module is upsampled by means of the deconvolutional layer and the residual module of the second neural network 102, to generate a fourth preset feature graph having a same resolution ratio as the fourth feature graph; or the feature graph processed by the DenseASPP module is upsampled by means of the deconvolutional layer and the residual module of the second neural network 102, to generate a third preset feature graph having a same resolution ratio as the third feature graph.

The third feature graph and the third preset feature graph are fused, to generate the first feature graph having a same resolution ratio as the third preset feature graph; the fourth feature graph and the fourth preset feature graph are fused, to generate the first feature graph having a same resolution ratio as the fourth preset feature graph; and the fifth feature graph and the fifth preset feature graph are fused, to generate the first feature graph having a same resolution ratio as the fifth preset feature graph; the third preset feature graph, the fourth preset feature graph, and the fifth preset feature graph respectively include the position of the lesion; and the position of the lesion is used for generating the position of the lesion in the first feature graph.

It should be explained that the DenseASPP module includes five dilated convolutions with different dilating rates combined and cascaded, and can extract the features of the lesions at different dimensions. The five dilated convolutions with different dilating rates are respectively: dilated convolutions with the dilating rate of d=3, dilated convolutions with the dilating rate of d=6, dilated convolutions with the dilating rate of d=12, dilated convolutions with the dilating rate of d=18, and dilated convolutions with the dilating rate of d=24.

The detection sub-network 103 may include: a first detection sub-network and a second detection sub-network. The first detection sub-network includes: a plurality of convolutional layers, and each of the plurality of convolutional layers is connected to a ReLU activation function. Similarly, the second detection sub-network includes: a plurality of convolutional layers, and each of the plurality of convolutional layers is connected to a ReLU activation function.

The first detection sub-network is used for detecting the second feature graph after dimension reduction on the first feature graph, to obtain the coordinate of the position of each lesion in the second feature graph by means of detection.

Specifically, the input second feature graph is processed by means of four cascaded convolutional layers in the first detection sub-network, wherein each convolutional layer includes a Y*Y convolution kernel; and by successively obtaining the coordinate (x1, y1) of the left upper corner of each lesion and the coordinate (x2, y2) of the right lower corner of the lesion, the position of each lesion in the second feature graph can be determined.

The second feature graph is detected by means of a second detection sub-network, to obtain a confidence score corresponding to each lesion in the second feature graph by means of detection.

Specifically, the input second feature graph is processed by means of four cascaded convolutional layers in the second detection sub-network, wherein each convolutional layer includes a Y*Y convolution kernel; and by successively obtaining the coordinate (x1, y1) of the left upper corner of each lesion and the coordinate (x2, y2) of the right lower corner of the lesion, the position of each lesion in the second feature graph can be determined.

It should be explained that the confidence score corresponding to the position in the embodiments of the present disclosure is the degree to which the authenticity that the position is a lesion believed by the user. For example, the confidence score of the position of a certain lesion may be 90%.

In view of the above, it may accurately detect lesion conditions of multiple parts in a patient body, and implement cancer preliminary assessment of a full body range of the patient.

It should be explained that before performing feature extraction on the first image to generate the first feature graph including the feature and position of the lesion, it further includes the following steps:

A pre-stored three-dimensional image including a plurality of lesion annotations is input into the first neural network; the lesion annotations are used for annotation of the lesions (for example, on one hand, the lesion is annotated by means of a frame, and on the other hand the coordinate of the position of the lesion is annotated); and a gradient descent method is used for respectively training each parameter of the first neural network, the second neural network, the first detection sub-network, and the second detection sub-network, where the position of each lesion in the plurality of lesions are output by the first detection sub-network.

It should be explained that, during the process of training each parameter using the gradient descent method, the gradient in the gradient descent method can be calculated by means of a counterpropagation method.

Or, a pre-stored three-dimensional image including a plurality of lesion annotations is input into the second neural network; the lesion annotations are used for annotation of the lesions; and the gradient descent method is used for respectively training each parameter of the second neural network, the first detection sub-network, and the second detection sub-network, where the position of each lesion in the plurality of lesions are output by the first detection sub-network.

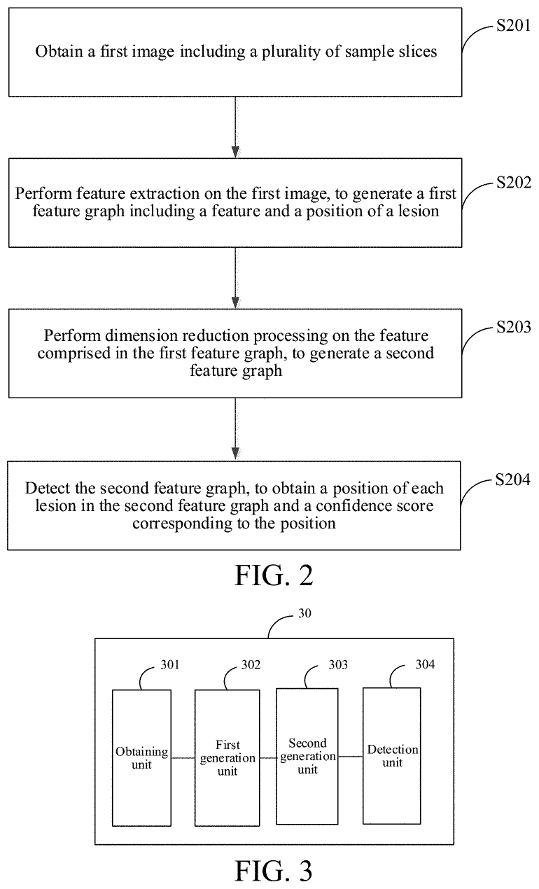

With reference to FIG. 2, it is a schematic flowchart of a lesion detection method provided by the present disclosure. In a possible implementation, the lesion detection method may be performed by an electronic device such as a terminal device or a server. The terminal device may be a User Equipment (UE), a mobile device, a user terminal, a terminal, a cordless phone, a Personal Digital Assistant (PDA), a handheld device, a computing device, a vehicle-mounted device, a wearable device, or the like. The method may be implemented by a processor by invoking computer-readable instructions stored in a memory. Alternatively, the method may be performed by a server.

As shown in FIG. 2, the method at least may include the following few steps:

S201, a first image including a plurality of sample slices is obtained, and the first image being a three-dimensional image including an X axis dimension, a Y axis dimension, and a Z axis dimension.

Specifically, in an optional implementation, an obtained CT image of a patient at a first sampling interval is resampled, to generate the first image including the plurality of sample slices. The CT image of the patient may include the fault layer number of 130; the thickness of each fault layer is 2.0 mm, and the first sampling interval at the X axis dimension and the Y axis dimension may be 2.0 mm.

In the embodiments of the present disclosure, the CT image of the patient is a scanning sequence including multiple fault layer numbers regarding the tissues or organs of the patient, and the fault layer number may be 130.

Lesion refers to a site of the lesion due to an action of pathogenic factors on tissues or organs of the patient, and is a part on a body suffered from pathological changes. For example, a part of a lung of a body is destroyed by tubercle bacillus, and then this part is a tuberculosis (TB) lesion.

It should be explained that the first image is a three-dimensional image including an X axis dimension, a Y axis dimension, and a Z axis dimension (that is, the first image is a three-dimensional image including an X axis dimension, a Y axis dimension, and a Z axis dimension constituted by N two-dimensional images including an X axis dimension and a Y axis dimension, and N is greater than or equal to 2; each two-dimensional image is a cross-sectional image of a tissue to be detected on a different position), for example, the first image may be a three-dimensional image of 512*512*9.

It should be explained that, before resampling the CT image, the following steps are further included:

the remaining background in the CT image is removed based on the threshold method.

S202, feature extraction is performed on the first image, to generate a first feature graph including a feature of a lesion; the first feature graph includes a three-dimensional feature including an X axis dimension, a Y axis dimension, and a Z axis dimension.

Specifically, performing feature extraction on the first image, to generate the first feature graph including the feature and position of the lesion may include, but not limited to, the following several conditions.

Condition 1: the first image is downsampled by means of a first neural network, to generate a third feature graph.

The third feature graph is downsampled by means of a residual module of the second neural network, to generate a fourth feature graph.

Features of lesions having different dimensions in the fourth feature graph are extracted by means of the DenseASPP module of the second neural network.

After processing by the DenseASPP module, a fourth preset feature graph having a same resolution ratio is generated as the fourth feature graph, and the feature graph processed by the DenseASPP module is upsampled by means of a deconvolutional layer and the residual module of the second neural network, to generate a third preset feature graph having a same resolution ratio as the third feature graph.

The third feature graph and the third preset feature graph are fused to generate a first feature graph having a same resolution ratio as the third preset feature graph from, and the four feature graph and the fourth preset feature graph are fused, to generate a first feature graph having a same resolution ratio as the fourth preset feature graph; the third preset feature graph and the fourth preset feature graph respectively include the position of the lesion; and the position of the lesion is used for generating the position of the lesion in the first feature graph.

Condition 2: the first image is downsampled by means of a residual module of the second neural network, to generate a fourth feature graph.

Features of lesions having different dimensions in the fourth feature graph are extracted by means of the DenseASPP module of the second neural network.

After processing by the DenseASPP module, the feature graph processed by the DenseASPP module is upsampled by means of the deconvolutional layer and the residual module of the second neural network, to generate the first preset feature graph having a same resolution ratio as the first image.

The first feature graph having a same resolution ratio as the first preset feature graph is generated from the first image and the first preset feature graph; the first preset feature graph includes the position of the lesion; and the position of the lesion is used for generating the position of the lesion in the first feature graph.

Condition 3: the first image is downsampled by means of a first neural network, to generate a third feature graph.

The third feature graph is downsampled by means of a residual module of the second neural network, to generate a fourth feature graph.

The fourth feature graph is downsampled by means of a residual module of the second neural network, to generate a fifth feature graph.

Features of lesions having different dimensions in the fourth feature graph are extracted by means of the DenseASPP module of the second neural network.

After processing by the DenseASPP module, a fifth preset feature graph having a same resolution ratio as the fifth feature graph is generated, and the feature graph processed by the DenseASPP module is upsampled by means of the deconvolutional layer and the residual module of the second neural network, to generate a fourth preset feature graph having a same resolution ratio as the fourth feature graph; or the feature graph processed by the DenseASPP module is upsampled by means of the deconvolutional layer and the residual module of the second neural network, to generate a third preset feature graph having a same resolution ratio as the third feature graph.

The third feature graph and the third preset feature graph are fused, to generate the first feature graph having a same resolution ratio as the third preset feature graph; the fourth feature graph and the fourth preset feature graph are fused, to generate the first feature graph having a same resolution ratio as the fourth preset feature graph; and the fifth feature graph and the fifth preset feature graph are fused, to generate the first feature graph having a same resolution ratio as the fifth preset feature graph; the third preset feature graph, the fourth preset feature graph, and the fifth preset feature graph respectively include the position of the lesion; and the position of the lesion is used for generating the position of the lesion in the first feature graph.

It should be explained that the first neural network includes a convolutional layer and a residual module cascaded with the convolutional layer.

The second neural network includes: a 3D U-Net network; the 3D U-Net network includes: a convolutional layer, a deconvolutional layer, a residual module, and the DenseASPP module.

The residual module may include: a convolutional layer, a BN layer, a ReLU activation function, and a maximum pooling layer.

Optionally, the second neural network is a plurality of stacked 3D U-Net networks. If the second neural network is a plurality of stacked 3D U-Net networks, the stability of the lesion detection system and the accuracy of the detection can be improved; the embodiments of the present disclosure do not limit the number of the 3D U-Net networks.

S203, dimension reduction processing is performed on the feature included in the first feature graph, to generate a second feature graph; and the second feature graph includes a two-dimensional feature of the X axis dimension and the Y axis dimension.

respectively combining a channel dimension and Z axis dimension of each feature in all features of the first feature graph, so that the dimension of each feature in all features of the first feature graph consists of the X axis dimension and the Y axis dimension; wherein the first feature graph with the dimension of each feature in all the features consisting of the X axis dimension and the Y axis dimension is the second feature graph. The first feature graph is a three-dimensional feature graph, while when output to the detection sub-network 103 for detection, it needs to be converted as two-dimensional, and therefore, the first feature graph requires to be subjected to dimension reduction.

It should be explained that the channel for a certain feature above represents the distribution data of a certain feature.

S204, the feature of the second feature graph is detected to obtain a feature of each lesion in the second feature graph and a confidence score corresponding to the position for displaying.

Specifically, the second feature graph is detected by means of a first detection sub-network, to obtain a coordinate of the position of each lesion in the second feature graph by means of detection.

Specifically, the input second feature graph is processed by means of multiple cascaded convolutional layers, wherein each convolutional layer includes a Y*Y convolution kernel; and by successively obtaining the coordinate (x1, y1) of the left upper corner of each lesion and the coordinate (x2, y2) of the right lower corner of the lesion, the position of each lesion in the second feature graph can be determined.

The second feature graph is detected by means of a second detection sub-network, to obtain a confidence score corresponding to each lesion in the second feature graph by means of detection.

Specifically, the input second feature graph is processed by means of multiple cascaded convolutional layers in the second detection sub-network, wherein each convolutional layer includes a Y*Y convolution kernel; and by successively obtaining the coordinate (x1, y1) of the left upper corner of each lesion and the coordinate (x2, y2) of the right lower corner of the lesion, the position of each lesion in the second feature graph can be determined.

In view of the above, the embodiments of the present disclosure may accurately detect lesion conditions of multiple parts in a patient body, and implement cancer preliminary assessment of a full body range of the patient.

It should be explained that before performing feature extraction on the first image to generate the first feature graph including the feature of the lesion, it further includes the following steps:

a pre-stored three-dimensional image including a plurality of lesion annotations is input into the first neural network; the lesion annotations are used for annotation of the lesions; and the gradient descent method is used for respectively training each parameter of the first neural network, the second neural network, the first detection sub-network, and the second detection sub-network, where the position of each lesion in the plurality of lesions are output by the first detection sub-network, or a three-dimensional image including a plurality of lesion annotations is input into the second neural network; the lesion annotations are used for annotation of the lesions; and the gradient descent method is used for respectively training each parameter of the second neural network, the first detection sub-network, and the second detection sub-network, where the position of each lesion in the plurality of lesions are output by the first detection sub-network.

In view of the above, in the present disclosure, first, a first image comprising a plurality of sample slices is obtained, and the first image is a three-dimensional image comprising an X axis dimension, a Y axis dimension, and a Z axis dimension. Furthermore, feature extraction is performed on the first image, to generate a first feature graph including a feature of a lesion; the first feature graph includes a three-dimensional feature including an X axis dimension, a Y axis dimension, and a Z axis dimension. Then, dimension reduction processing is performed on the feature included in the first feature graph, to generate a second feature graph; and the second feature graph includes a two-dimensional feature of the X axis dimension and the Y axis dimension. Finally, the feature of the second feature graph is detected to obtain a position of each lesion in the second feature graph and a confidence score corresponding to the position. Using the embodiments of the present disclosure may accurately detect lesion conditions of multiple parts in a patient body, and implement cancer preliminary assessment of a full body range of the patient.

It can be understood that for the related definition and explanations that are not provided in the method embodiment in FIG. 2, please refer to the embodiment in FIG. 1, and it would not be repeated herein.

With reference to FIG. 3, it is a lesion detection apparatus provided by the present disclosure. As shown in FIG. 3, the lesion detection apparatus 30 includes: an obtaining unit 301, a first generation unit 302, a second generation unit 303, and a detection unit 304, where

the obtaining unit 301 is configured to obtain a first image comprising a plurality of sample slices, the first image being a three-dimensional image comprising an X axis dimension, a Y axis dimension, and a Z axis dimension;

the first generation unit 302 is configured to perform feature extraction on the first image, to generate a first feature graph comprising a feature and a position of a lesion; the first feature graph comprising a three-dimensional feature comprising an X axis dimension, a Y axis dimension, and a Z axis dimension;

the second generation unit 303 is configured to perform dimension reduction processing on the feature comprised in the first feature graph, to generate a second feature graph; the second feature graph comprising a two-dimensional feature of the X axis dimension and the Y axis dimension; and

the detection unit 304 is configured to detect the second feature graph, to obtain a position of each lesion in the second feature graph and a confidence score corresponding to the position.

The obtaining unit 302 is specifically configured to:

resample an obtained CT image of a patient at a first time interval, to generate the first image comprising the plurality of sample slices.

The first generation unit 303 may specifically be used for the following three conditions:

Condition 1: the first image is downsampled by means of a first neural network, to generate a third feature graph.

The third feature graph is downsampled by means of a residual module of the second neural network, to generate a fourth feature graph.

Features of lesions having different dimensions in the fourth feature graph are extracted by means of the DenseASPP module of the second neural network.

After processing by the DenseASPP module, a fourth preset feature graph having a same resolution ratio is generated as the fourth feature graph, and the feature graph processed by the DenseASPP module is upsampled by means of a deconvolutional layer and the residual module of the second neural network, to generate a third preset feature graph having a same resolution ratio as the third feature graph.

The third feature graph and the third preset feature graph are fused to generate a first feature graph having a same resolution ratio as the third preset feature graph from, and the four feature graph and the fourth preset feature graph are fused, to generate a first feature graph having a same resolution ratio as the fourth preset feature graph; the third preset feature graph and the fourth preset feature graph respectively include the position of the lesion; and the position of the lesion is used for generating the position of the lesion in the first feature graph.

Condition 2: the first image is downsampled by means of a residual module of the second neural network, to generate a fourth feature graph.

Features of lesions having different dimensions in the fourth feature graph are extracted by means of the DenseASPP module of the second neural network.

After processing by the DenseASPP module, the feature graph processed by the DenseASPP module is upsampled by means of the deconvolutional layer and the residual module of the second neural network, to generate the first preset feature graph having a same resolution ratio as the first image.

The first feature graph having a same resolution ratio as the first preset feature graph is generated from the first image and the first preset feature graph; the first preset feature graph includes the position of the lesion; and the position of the lesion is used for generating the position of the lesion in the first feature graph.

Condition 3: the first image is downsampled by means of a first neural network, to generate a third feature graph.

The third feature graph is downsampled by means of a residual module of the second neural network, to generate a fourth feature graph.

The fourth feature graph is downsampled by means of a residual module of the second neural network, to generate a fifth feature graph.

Features of lesions having different dimensions in the fourth feature graph are extracted by means of the DenseASPP module of the second neural network.

After processing by the DenseASPP module, a fifth preset feature graph having a same resolution ratio as the fifth feature graph is generated, and the feature graph processed by the DenseASPP module is upsampled by means of the deconvolutional layer and the residual module of the second neural network, to generate a fourth preset feature graph having a same resolution ratio as the fourth feature graph; or the feature graph processed by the DenseASPP module is upsampled by means of the deconvolutional layer and the residual module of the second neural network, to generate a third preset feature graph having a same resolution ratio as the third feature graph.

The third feature graph and the third preset feature graph are fused, to generate the first feature graph having a same resolution ratio as the third preset feature graph; the fourth feature graph and the fourth preset feature graph are fused, to generate the first feature graph having a same resolution ratio as the fourth preset feature graph; and the fifth feature graph and the fifth preset feature graph are fused, to generate the first feature graph having a same resolution ratio as the fifth preset feature graph; the third preset feature graph, the fourth preset feature graph, and the fifth preset feature graph respectively include the position of the lesion; and the position of the lesion is used for generating the position of the lesion in the first feature graph.

It should be explained that the first neural network includes a convolutional layer and a residual module cascaded with the convolutional layer.

The second neural network includes: a 3D U-Net network; the 3D U-Net network may include: a convolutional layer, a deconvolutional layer, a residual module, and the DenseASPP module.

Optionally, the second neural network may include a plurality of stacked 3D U-Net networks. Multiple detections of the 3D U-net networks may improve the accuracy of the detection; the embodiments of the present disclosure only exemplify the number of the 3D U-net network.

It should be explained that the residual module may include: a convolutional layer, a BN layer, a ReLU activation function, and a maximum pooling layer.

The third feature unit 304 is specifically used for: respectively combining a channel dimension and Z axis dimension of each feature in all features of the first feature graph, so that the dimension of each feature in all features of the first feature graph consists of the X axis dimension and the Y axis dimension; wherein the first feature graph with the dimension of each feature in all the features consisting of the X axis dimension and the Y axis dimension is the second feature graph.

The detection unit 305 is specifically configured to:

detect the second feature graph by means of a first detection sub-network, to obtain a coordinate of the position of each lesion in the second feature graph by means of detection.

The second feature graph is detected by means of a second detection sub-network, to obtain a confidence score corresponding to each lesion in the second feature graph by means of detection.

It should be explained that the first detection sub-network includes: a plurality of convolutional layers, and each of the plurality of convolutional layers is connected to a ReLU activation function.

The second detection sub-network includes: a plurality of convolutional layers, and each of the plurality of convolutional layers is connected to a ReLU activation function.

The lesion detection apparatus 30 includes: an obtaining unit 301, a first generation unit 302, a second generation unit 303, and a detection unit 304, and in addition, it further includes a display unit.

The display unit is specifically configured to display the position of the lesion and the confidence score of the position detected by the detection unit 304.

The lesion detection apparatus 30 includes: an obtaining unit 301, a first generation unit 302, a second generation unit 303, and a detection unit 304, and in addition, it further includes a training unit.

The training unit is specifically configured to:

before the first generation unit performs feature extraction on the first image to generate a first feature graph including a feature and a position of a lesion, input a pre-stored three-dimensional image including a plurality of lesion annotations into the first neural network; the lesion annotations being used for annotation of the lesions; and use a gradient descent method to respectively train each parameter of the first neural network, the second neural network, the first detection sub-network, and the second detection sub-network, where the position of each lesion in the plurality of lesions are output by the first detection sub-network, or

before the first generation unit performs feature extraction on the first image to generate a first feature graph including a feature and a position of a lesion, input a three-dimensional image including a plurality of lesion annotations into the second neural network; the lesion annotations being used for annotation of the lesions; and use a gradient descent method to respectively train each parameter of the second neural network, the first detection sub-network, and the second detection sub-network.

It should be understood that the lesion detection apparatus 30 is only an example provided by the embodiments of the present disclosure, and the lesion detection apparatus 30 may include components more or less than the shown ones; two or more components may be combined, or it may be implemented by having different configurations for the components.

It should be understood that for the specific implementations of the functional blocks included in the lesion detection apparatus 30 in FIG. 3, please refer to the method embodiment according to FIG. 2, and it would not be repeated herein.

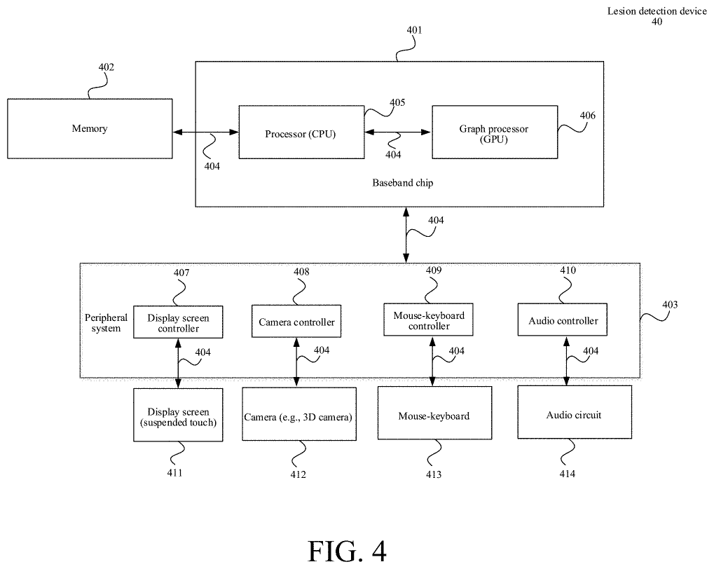

FIG. 4 is a structural schematic diagram of a lesion detection apparatus provided by the present disclosure. In the embodiments of the present disclosure, the lesion detection device may include various devices such as a mobile phone, a tablet computer, a Personal Digital Assistant (PDA), a Mobile Internet Device (MID, and a smart wearing device (e.g., a smart watch and a smart bracelet). The embodiments of the present disclosure do not make any limitation. As shown in FIG. 4, the lesion detection device 40 may include: a baseband chip 401, a memory 402 (one or more computer readable storage media), and a peripheral system 403. These components may communication over one or more communication buses 404.

The baseband chip 401 includes: one or more CPUs 405 and one or more GPUs 406. The GPU 406 may be configured to processing an input normal chartlet.

The memory 402 is coupled with the processor 405, for storing various software programs and/or multiple groups of instructions. In specific implementations, the memory 402 may include a high-speed random-access memory, and may also include a nonvolatile memory, such as one or more disk storage devices, flash memory devices, or other nonvolatile solid-state storage devices. The memory 402 may store an operation system (referred to as a system for short hereinafter), for example, an embedded operation system such as ANDROID, IOS, WINDOWS, or LINUX. The memory 402 may store a network communication program; the network communication program may be used for communicating with one or more attachment devices, one or more devices, and one or more network devices. The memory 402 may further store a user interface program; the user interface program may vividly display the content of the application program by means of a graphic operation interface and may receive the control operation of the corresponding application program of the user by means of input controls such as a menu, a dialog box, and a key.

It should be understood that the memory 402 may be used for storing the program codes for implementing the lesion detection method.

It could be understood that the processor 405 may be used for invoking the program codes for executing the lesion detection method stored in the memory 402.

The memory 402 may further store one or more application programs. As shown in FIG. 4, the application programs may include, a social application program (e.g., Facebook), an image management application program (e.g., a photo album), a map type application program (e.g., a Google map), a browser (e.g., Safari, Google Chrome), and the like.

The peripheral system 403 is mainly used for implementing the interaction function between the lesion detection device 40 and the user/external environment, and mainly includes an input/output device of the lesion detection device 40. In specific implementations, the peripheral system 403 may include: a display screen controller 407, a camera controller 408, a mouse-keyboard controller 409, and an audio controller 410. Each controller may be coupled with the corresponding peripheral device respectively (e.g., a display screen 411, a camera 412, a mouse-keyboard 413, and an audio circuit 414). In some embodiments, the display screen may be a display screen configured with a self-capacitive suspension touch panel, and may also be a display screen configured with an infrared suspension touch panel. In some embodiments, the camera 412 may be a 3D camera. It should be explained that the peripheral system 403 may further include other I/O external devices.

It could be understood that the display screen 411 may be used for displaying the detected lesion position and the confidence score of the position.

It should be understood that the lesion detection device 40 is only an example provided by the embodiments of the present disclosure, and the lesion detection device 40 may include components more or less than the shown ones; two or more components may be combined, or it may be implemented by having different configurations for the components.

It should be understood that for the specific implementations of the functional blocks included in the lesion detection device 40 in FIG. 4, please refer to the method embodiment according to FIG. 2, and it would not be repeated herein.

The present disclosure provides a computer readable storage medium; the computer readable storage medium stores a computer program; the computer program is implemented when executed by a processor.

The computer readable storage medium may be an internal storage unit of the device according to any of the preceding embodiments, for example, a hard disk or memory of the device. The computer readable storage medium may also be an external storage device of the device, such as an insertion-type hard disk drive, a Smart Media Card (SMC), a Secure Digital (SD) card, and a flash card configured on the device. Furthermore, the computer readable storage medium can further include both the internal storage unit and the external storage device of the device. The computer readable storage medium is used for storing a computer program and other programs and data required by the device. The computer readable storage medium can further be used for temporarily store output data or data to be output.

The present disclosure further provides a computer program product; the computer program product includes an instantaneous computer readable storage medium storing a computer program; the computer program can be operated to enable the computer to execute same or all steps of any method recited in the aforementioned method embodiments. The computer program product may be a software package; the computer includes an electronic device.

Persons skilled in the art can understand that the individual exemplary units and arithmetic steps that are described in conjunction with the embodiments disclosed herein are able to be implemented in the electronic hardware, the computer software or a combination thereof. For describing the interchangeability between the hardware and the software clearly, the components and the steps of each example have been described according to the function generally in the description above. Whether these functions are performed by hardware or software depends on the particular applications and design constraint conditions of the technical solutions. For each specific application, the described functions can be implemented by a person skilled in the art using different methods, but this implementation should not be considered to go beyond the scope of the present disclosure.

A person skilled in the art can clearly understand that for convenience and brevity of description, reference is made to corresponding process descriptions in the foregoing method embodiments for the specific working processes of the device and the units described above, and details are not described herein again.

It should be understood that the disclosed device and method in the embodiments provided in the present disclosure may be implemented by means of other modes. For example, the compositions and steps of each example are described. Whether these functions are performed by hardware or software depends on the particular applications and design constraint conditions of the technical solutions. For each specific application, the described functions can be implemented by a person skilled in the art using different methods, but this implementation should not be considered to go beyond the scope of the present disclosure.

The device embodiments described above are merely exemplary. For example, the unit division is merely logical function division and may be actually implemented by other division modes. For example, a plurality of units or components may be combined or integrated into another system, or some features may be ignored or not performed. In addition, the displayed or discussed mutual couplings or direct couplings or communication connections may be implemented by means of some interfaces, apparatuses or units. The indirect couplings or communication connections between the apparatuses or units may be implemented in electronic, mechanical, or other forms.

The units described as separate parts may or may not be physically separate, and the parts displayed as units may or may not be physical units, may be located at one position, or may be distributed on a plurality of network units. A part of or all of the units may be selected according to actual needs to achieve the objectives of the solutions of the embodiments of the present disclosure.

In addition, functional units in the embodiments of the present disclosure may be integrated into one processing unit, or each of the units may exist alone physically, or two or more units may be integrated into one unit. The integrated unit may be implemented in a form of hardware and may also be implemented in a form of a software functional unit.

When the integrated module/unit is implemented in a form of a software functional unit and sold or used as an independent product, the integrated module/unit may be stored in a computer readable storage medium. Based on such an understanding, the technical solutions of the present disclosure or a part thereof contributing to the prior art may be essentially embodied in the form of a software product. The computer software product is stored in one storage medium and includes several instructions so that one computer device (which may be a personal computer, a target blockchain node device, a network device, and the like) implements all or part of steps of the method in the embodiments of the present disclosure. Moreover, the preceding storage medium includes: media having program codes stored such as a USB flash drive, a mobile hard disk drive, a Read-only Memory (ROM), a floppy disk, and an optical disc.

The descriptions above only provide specific implementations of this disclosure. However, the scope of protection of this disclosure is not limited thereto. Within the technical scope disclosed by this disclosure, various equivalent amendments or substitutions that can be easily conceived of by those skilled in the art should all fall within the scope of protection of this disclosure. Therefore, the scope of protection of the present disclosure should be subjected to the scope of protection of the claims.

What is claimed is:

1. A lesion detection method, comprising:
obtaining a first image comprising a plurality of sample slices, the first image being a three-dimensional image comprising an X axis dimension, a Y axis dimension, and a Z axis dimension;
performing feature extraction on the first image, to generate a first feature graph comprising a feature and a position of a lesion; the first feature graph comprising a three-dimensional feature comprising an X axis dimension, a Y axis dimension, and a Z axis dimension;
performing dimension reduction processing on the feature comprised in the first feature graph, to generate a second feature graph; the second feature graph comprising a two-dimensional feature of the X axis dimension and the Y axis dimension; and
detecting the second feature graph, to obtain a position of each lesion in the second feature graph and a confidence score corresponding to the position.

2. The method according to claim 1, wherein obtaining the first image comprising the plurality of sample slices comprises:
resampling an obtained CT image of a patient at a first sampling interval, to generate the first image comprising the plurality of sample slices.

3. The method according to claim 1, wherein performing feature extraction on the first image, to generate the first feature graph comprising the feature and the position of the lesion, comprises:
downsampling the first image by means of a first neural network, to generate a third feature graph;
downsampling the third feature graph by means of a residual module of a second neural network, to generate a fourth feature graph;
extracting features of lesions having different dimensions in the fourth feature graph by means of a DenseASPP module of the second neural network;
after processing by the DenseASPP module, generating a fourth preset feature graph having a same resolution ratio as the fourth feature graph, and upsampling the feature graph processed by the DenseASPP module by means of a deconvolutional layer and the residual module of the second neural network, to generate a third preset feature graph having a same resolution ratio as the third feature graph; and
fusing the third feature graph and the third preset feature graph, to generate a first feature graph having a same resolution ratio as the third preset feature graph, and fusing the four feature graph and the fourth preset feature graph, to generate a first feature graph having a same resolution ratio as the fourth preset feature graph; the third preset feature graph and the fourth preset feature graph respectively comprising the position of the lesion; and the position of the lesion being used for generating the position of the lesion in the first feature graph.

4. The method according to claim 1, wherein performing feature extraction on the first image, to generate the first feature graph comprising the feature and the position of the lesion, comprises:
downsampling the first image by means of a residual module of a second neural network, to generate a fourth feature graph;
extracting features of lesions having different dimensions in the fourth feature graph by means of a DenseASPP module of the second neural network;
after processing by the DenseASPP module, upsampling the feature graph processed by the DenseASPP module by means of a deconvolutional layer and the residual module of the second neural network, to generate a first preset feature graph having a same resolution ratio as the first image; and
fusing the first image and the first preset feature graph, to generate the first feature graph having a same resolution ratio as the first preset feature graph; the first preset feature graph comprising the position of the lesion; and the position of the lesion being used for generating the position of the lesion in the first feature graph.

5. The method according to claim 1, wherein performing feature extraction on the first image, to generate the first feature graph comprising the feature and the position of the lesion, comprises:
downsampling the first image by means of a first neural network, to generate a third feature graph having the resolution ratio less than the first image;
downsampling the third feature graph by means of a residual module of a second neural network, to generate a fourth feature graph;
downsampling the fourth feature graph by means of the residual module of the second neural network, to generate a fifth feature graph having a resolution ratio less than the fourth feature graph;
extracting features of lesions having different dimensions in the fifth feature graph by means of a DenseASPP module of the second neural network;
after processing by the DenseASPP module, generating a fifth preset feature graph having a same resolution ratio as the fifth feature graph, and upsampling the feature graph processed by the DenseASPP module by means of a deconvolutional layer and the residual module of the second neural network, to generate a fourth preset feature graph having a same resolution ratio as the fourth feature graph; or upsampling the feature graph processed by the DenseASPP module by means of the deconvolutional layer and the residual module of the second neural network, to generate a third preset feature graph having a same resolution ratio as the third feature graph; and
fusing the third feature graph and the third preset feature graph, to generate the first feature graph having a same resolution ratio as the third preset feature graph; fusing the fourth feature graph and the fourth preset feature graph, to generate the first feature graph having a same resolution ratio as the fourth preset feature graph; and fusing the fifth feature graph and the fifth preset feature graph, to generate the first feature graph having a same resolution ratio as the fifth preset feature graph; the third preset feature graph, the fourth preset feature graph, and the fifth preset feature graph respectively comprising the position of the lesion; and the position of the lesion being used for generating the position of the lesion in the first feature graph.

6. The method according to claim 3, wherein
the first neural network comprises: a convolutional layer and a residual module cascaded with the convolutional layer;
the second neural network is a plurality of stacked 3D U-Net network; the 3D U-Net network comprises: a convolutional layer, the deconvolutional layer, the residual module, and the DenseASPP module; and
the residual module comprises: a convolutional layer, a batch normalization layer, a ReLU activation function, and a maximum pooling layer.

7. The method according to claim 1, wherein performing dimension reduction processing on the feature comprised in the first feature graph, to generate the second feature graph, comprises:
respectively combining a channel dimension and Z axis dimension of each feature in all features of the first feature graph, so that the dimension of each feature in all features of the first feature graph consists of the X axis dimension and the Y axis dimension; wherein the first feature graph with the dimension of each feature in all the features consisting of the X axis dimension and the Y axis dimension is the second feature graph, or
detecting the second feature graph comprises:
detecting the second feature graph by means of a first detection sub-network, to obtain a coordinate of the position of each lesion in the second feature graph by means of detection; and
detecting the second feature graph by means of a second detection sub-network, to obtain a confidence score corresponding to each lesion in the second feature graph by means of detection.

8. The method according to claim 7, wherein
the first detection sub-network comprises: a plurality of convolutional layers, each of the plurality of convolutional layers being connected to a ReLU activation function; and
the second detection sub-network comprises: a plurality of convolutional layers, each of the plurality of convolutional layers being connected to a ReLU activation function.

9. The method according to claim 1, before performing feature extraction on the first image, to generate the first feature graph comprising the feature and the position of the lesion, further comprising:
inputting a pre-stored three-dimensional image comprising a plurality of lesion annotations into a first neural network; the lesion annotations being used for annotation of the lesions; and using a gradient descent method to respectively train each parameter of the first neural network, a second neural network, a first detection sub-network, and a second detection sub-network, wherein the position of each lesion in the plurality of lesions are output by the first detection sub-network; or
inputting a three-dimensional image comprising a plurality of lesion annotations into the second neural network; the lesion annotations being used for annotation of the lesions; and using a gradient descent method to respectively train each parameter of the second neural network, the first detection sub-network, and the second detection sub-network, wherein the position of each lesion in the plurality of lesions is output by the first detection sub-network.

10. A lesion detection apparatus, comprising:
a processor; and
a memory storing processor-executable codes which, when executed by the processor, cause the processor to:
obtain a first image comprising a plurality of sample slices, the first image being a three-dimensional image comprising an X axis dimension, a Y axis dimension, and a Z axis dimension;
perform feature extraction on the first image, to generate a first feature graph comprising a feature and a position of a lesion; the first feature graph comprising a three-dimensional feature comprising an X axis dimension, a Y axis dimension, and a Z axis dimension;
perform dimension reduction processing on the feature comprised in the first feature graph, to generate a second feature graph; the second feature graph comprising a two-dimensional feature of the X axis dimension and the Y axis dimension; and
detect the second feature graph, to obtain a position of each lesion in the second feature graph and a confidence score corresponding to the position.

11. The apparatus according to claim 10, wherein obtaining the first image comprising the plurality of sample slices comprises:
resampling an obtained CT image of a patient at a first sampling interval, to generate the first image comprising the plurality of sample slices.

12. The apparatus according to claim 10, wherein performing feature extraction on the first image, to generate the first feature graph comprising the feature and the position of the lesion, comprises:
downsampling the first image by means of a first neural network, to generate a third feature graph;
downsampling the third feature graph by means of a residual module of a second neural network, to generate a fourth feature graph;
extracting features of lesions having different dimensions in the fourth feature graph by means of a DenseASPP module of the second neural network;
after processing by the DenseASPP module, generating a fourth preset feature graph having a same resolution ratio as the fourth feature graph, and upsampling the feature graph processed by the DenseASPP module by means of a deconvolutional layer and the residual module of the second neural network, to generate a third preset feature graph having a same resolution ratio as the third feature graph; and
fusing the third feature graph and the third preset feature graph, to generate a first feature graph having a same resolution ratio as the third preset feature graph, and fusing the four feature graph and the fourth preset feature graph, to generate a first feature graph having a same resolution ratio as the fourth preset feature graph; the third preset feature graph and the fourth preset feature graph respectively comprising the position of the lesion; and the position of the lesion being used for generating the position of the lesion in the first feature graph.

13. The apparatus according to claim 10, wherein performing feature extraction on the first image, to generate the first feature graph comprising the feature and the position of the lesion, comprises:
downsampling the first image by means of a residual module of a second neural network, to generate a fourth feature graph;
extracting features of lesions having different dimensions in the fourth feature graph by means of a DenseASPP module of the second neural network;
after processing by the DenseASPP module, upsampling the feature graph processed by the DenseASPP module by means of a deconvolutional layer and the residual module of the second neural network, to generate a first preset feature graph having a same resolution ratio as the first image; and
fusing the first image and the first preset feature graph, to generate the first feature graph having a same resolution ratio as the first preset feature graph; the first preset feature graph comprising the position of the lesion; and the position of the lesion being used for generating the position of the lesion in the first feature graph.

14. The apparatus according to claim 10, wherein performing feature extraction on the first image, to generate the first feature graph comprising the feature and the position of the lesion, comprises:
downsampling the first image by means of a first neural network, to generate a third feature graph having the resolution ratio less than the first image;
downsampling the third feature graph by means of a residual module of a second neural network, to generate a fourth feature graph;
downsampling the fourth feature graph by means of a residual module of the second neural network, to generate a fifth feature graph having a resolution ratio less than the fourth feature graph;
extracting features of lesions having different dimensions in the fifth feature graph by means of a DenseASPP module of the second neural network;
after processing by the DenseASPP module, generating a fifth preset feature graph having a same resolution ratio as the fifth feature graph, and upsampling the feature graph processed by the DenseASPP module by means of a deconvolutional layer and the residual module of the second neural network, to generate a fourth preset feature graph having a same resolution ratio as the fourth feature graph; or upsampling the feature graph processed by the DenseASPP module by means of the deconvolutional layer and the residual module of the second neural network, to generate a third preset feature graph having a same resolution ratio as the third feature graph; and
fusing the third feature graph and the third preset feature graph, to generate the first feature graph having a same resolution ratio as the third preset feature graph; fusing the fourth feature graph and the fourth preset feature graph, to generate the first feature graph having a same resolution ratio as the fourth preset feature graph; and fusing the fifth feature graph and the fifth preset feature graph, to generate the first feature graph having a same resolution ratio as the fifth preset feature graph; the third preset feature graph, the fourth preset feature graph, and the fifth preset feature graph respectively comprising the position of the lesion; and the position of the lesion being used for generating the position of the lesion in the first feature graph.

15. The apparatus according to claim 12, wherein
the first neural network comprises: a convolutional layer and a residual module cascaded with the convolutional layer;
the second neural network is a plurality of stacked 3D U-Net network; the 3D U-Net network comprises: a convolutional layer, the deconvolutional layer, the residual module, and the DenseASPP module; and
the residual module comprises: a convolutional layer, a batch normalization layer, a ReLU activation function, and a maximum pooling layer.

16. The apparatus according to claim 10, wherein
performing dimension reduction processing on the feature comprised in the first feature graph, to generate the second feature graph, comprises:
respectively combining a channel dimension and Z axis dimension of each feature in all features of the first feature graph, so that the dimension of each feature in all features of the first feature graph consists of the X axis dimension and the Y axis dimension; wherein the first feature graph with the dimension of each feature in all the features consisting of the X axis dimension and the Y axis dimension is the second feature graph, or
detecting the second feature graph comprises:
detecting the second feature graph by means of a first detection sub-network, to obtain a coordinate of the position of each lesion in the second feature graph by means of detection; and
detecting the second feature graph by means of a second detection sub-network, to obtain a confidence score corresponding to each lesion in the second feature graph by means of detection.

17. The apparatus according to claim 16, wherein
the first detection sub-network comprises: a plurality of convolutional layers, each of the plurality of convolutional layers being connected to a ReLU activation function; and
the second detection sub-network comprises: a plurality of convolutional layers, each of the plurality of convolutional layers being connected to a ReLU activation function.

18. The apparatus according to claim 10, wherein before performing feature extraction on the first image, to generate the first feature graph comprising the feature and the position of the lesion, the processor is further caused to:
input a pre-stored three-dimensional image comprising a plurality of lesion annotations into a first neural network; the lesion annotations being used for annotation of the lesions; and use a gradient descent method to respectively train each parameter of the first neural network, a second neural network, a first detection sub-network, and a second detection sub-network, wherein the position of each lesion in the plurality of lesions are output by the first detection sub-network; or
input a three-dimensional image comprising a plurality of lesion annotations into the second neural network; the lesion annotations being used for annotation of the lesions; and use a gradient descent method to respectively train each parameter of the second neural network, the first detection sub-network, and the second detection sub-network, wherein the position of each lesion in the plurality of lesions are output by the first detection sub-network.

19. A lesion detection device, comprising: a display, a memory, and a processor coupled with the memory, wherein the display is configured to display a position of a lesion and a confidence score corresponding to the position, the memory is configured to store application program codes, and the processor is configured to invoke the program codes, to execute the lesion detection method according to claim 1.

20. A non-transitory computer readable storage medium, wherein the computer storage medium stores a computer program, the computer program comprises program instructions, and when the program instructions are executed by a processor, the processor executes a lesion detection method comprising:
obtaining a first image comprising a plurality of sample slices, the first image being a three-dimensional image comprising an X axis dimension, a Y axis dimension, and a Z axis dimension;
performing feature extraction on the first image, to generate a first feature graph comprising a feature and a position of a lesion; the first feature graph comprising a three-dimensional feature comprising an X axis dimension, a Y axis dimension, and a Z axis dimension;
performing dimension reduction processing on the feature comprised in the first feature graph, to generate a second feature graph; the second feature graph comprising a two-dimensional feature of the X axis dimension and the Y axis dimension; and
detecting the second feature graph, to obtain a position of each lesion in the second feature graph and a confidence score corresponding to the position.
